(19) United States
(12) Patent Application Publication
(10) Pub. No.: US 2026/0306835 A1
(43) Pub. Date: Oct. 8, 2026

(54) LV FLAT ELECTRODE

(71) Applicant: Berlin HEALS GmbH, Berlin (DE)

(72) Inventor: Thomas Töllner, Ketzin (DE)

(21) Appl. No.: 19/474,966

(22) PCT Filed: Apr. 11, 2024
(86) PCT No.: PCT/EP2024/059874

§ 371 Date: Oct. 13, 2025

(30) Foreign Application Priority Data
Apr. 14, 2023 (EP) 23168101.6

(51) Int. Cl. A61N 1/05; H01B 5/14

(52) U.S. Cl. CPC A61N 1/0597; A61N 1/059; H01B 5/14

(57) ABSTRACT

The present invention provides an electrode, specifically an implantable flat electrode (1), useful for the treatment of organic tissue using an electrical current, comprising laminated first and second insulating layers (2, 3) comprising an elastic, biocompatible and biostable material, the second insulating (3) layer having at least one opening (3c), a flexible conductive material (4) sandwiched between the first and second insulating layers (2, 3), wherein the flexible conductive material (4) comprises an edge portion (4a) embedded into the first insulating layer (2) and covered by the second insulating layer (3), and a center portion (4b) exposed through the at least one opening (3c) of the second insulating layer (3) and not fixed to the first insulating layer (2), two or more pairs of suture holes (2a, 3a) provided in circumferential edge portions (2b, 3b) of the first and second insulating layers (2, 3), wherein the edge portions (2b, 3b) portions are laminated onto each other without the conductive material (4) sandwiched in between, and 9-7 an electrical connector (6) electrically coupled to the conductive material (4).

### TECHNICAL FIELD

The present invention relates to an electrode, specifically an implantable flat electrode, useful for the treatment of organic tissue using an electrical current comprising at least two insulating layers, a conductive material, an electrical connector as well as a process of manufacturing of a flat electrode.

### TECHNOLOGICAL BACKGROUND

First epicardial flat electrodes (hereinafter may also be referred to as “patch lead”) have been commonly used for Implantable Cardioverter Defibrillators (ICD's) starting in the early 1990's. Typical flat electrodes use silicones as the non-conductive lead part and titanium or platinum alloys as conductive material. These early electrodes have not been designed for direct current application and usually have an insufficiently small electrically active surface.

Flat electrodes for the treatment of organic tissue have been known from WO 2006/106132 A1, US 2010/152864 A1, and WO 2007/070579 A2. WO 2006/106132 A1 describes an electrode for treating organic tissue by means of direct current. US 2010/152864 A1 describes an implant for use on an organic tissue, comprising an electrical stimulation system, in which the control is suitable for limiting or controlling the current density at the implant-bone interface. WO 2007/070579 A2 describes an implant for stimulating the regeneration of damaged organic tissue, in which a direct current is applied near the damaged sites at a level sufficient to induce regeneration, without applying a current level at which tissue toxicity occurs.

Such flat electrodes usually comprise silicone materials and are being attached to the target tissue by suturing, clamping, stapling or by any other suitable method such that they fit closely to the surface of the target tissue. As fastening means, flat electrodes comprise, for example, suture holes integrated in the silicon material, which are usually arranged along the electrode edge and enable suturing to the target tissue.

In the case in which suture holes in the silicone material were utilized to fix electrodes and the silicone material is subjected to excess tensile load by the surgical suture, they may deform or even tear out entirely as silicone materials are very flexible but have only a low tensile strength. In these cases, the conductive electrode material of the implanted flat electrode may detach from the tissue partly or entirely, so that the treatment of the target tissue cannot be maintained most optimal.

Flat electrodes for the treatment of organic tissue are usually manufactured by either molding the conductive material with liquid silicone, having one side of the conductive material exposed, or by bonding the conductive material to a solid silicone layer with a suitable bonding agent. Both methods produce flat electrodes, in which the conductive material is connected to a single silicone layer also intended for attaching the flat electrode, usually in a part thereof in which the conductive material is not present. Due to the single-layer silicone structure, the silicone layer part intended for attaching, e.g. the circumferential edge area, of the flat electrode has weaknesses for a stable and reliable tissue attachment.

On the other hand, high flexibility and low stiffness in the electrically conductive part of the flat electrode is desirable in order to closely conform to the surface of the target organic tissue, which often has an uneven surface. It has been observed that flat electrodes which are too flexible and less rigid—especial in the circumferential area—can deform and displace after a certain period after implantation. The deformations and displacements can be caused by torn sutures and/or a biological reaction such as fibrosis. Deformations and displacements of electrodes can result in limited contact to the target tissue, so that the respective organ cannot be treated effectively.

Thus, there is a need for a flat electrode with an increased reinforcement against detachment due to tear-out and an increased circumferential rigidity against deformations after implantation while keeping the conductive part of the electrode area flexible to properly conform to an organ to be treated.

### SUMMARY OF THE INVENTION

In order to address the need as explained above, it is an object of the present invention to provide a flat electrode having a lower risk of detachment and deformation after implantation and enabling effective treatment of a treating organic tissue, and to provide a process of manufacturing of said flat electrodes.

In a first aspect of the present invention, the flat electrode comprises laminated first and second insulating layers comprising an elastic, biocompatible and biostable material, the second insulating layer having at least one opening, a flexible conductive material sandwiched between the first and second insulating layer, wherein the conductive material comprises an edge portion embedded into the first insulating layer and covered by the second insulating layer, and a center portion exposed through the at least one opening of the second insulating layer and not fixed to the first insulating layer, two or more pairs of suture holes provided in a circumferential edge portion of the first and second insulating layers, wherein the edge portions are laminated onto each other without the conductive material sandwiched in between, and an electrical connector electrically coupled to the conductive material. The design performs with an increased form stability against deformations in consequence of body reactions while being flexible and it provides reinforced fastening means to prevent a long-term migration of the electrode. This enhances the efficacy and safety of a treatment of organic tissue using electrical current.

According to an embodiment of the first aspect, the conductive material comprises a metallic mesh, preferably wherein the metallic mesh comprises platinum, more preferably a platinum-iridium alloy. The platinum-iridium alloy preferably has an alloy proportion of platinum-iridium of 70:30 to 99:1, more preferably of 80:20 to 95:15, most preferably of 88:12 to 92:8.

According to yet another embodiment of the first aspect, wherein the conductive material has a waist shape in plan view comprising in the longitudinal direction thereof two distal portions connected by a waist portion therebetween, the ratio of the width perpendicular to the longitudinal direction of the distal portion to the waist portion is from 1.5:1 to 7:1, preferably from 2:1 to 6:1, more preferably from 3:1 to 5:1.

According to yet another embodiment of the first aspect, the flat electrode comprises a frame arranged between the first and second insulating layer, wherein the frame comprises a plurality of protrusions each protruding from the frame and extending in parallel to the surface of the insulating layers between a pair of adjacent suture holes. Optionally, the frame is separated from the conductive material by at least one additional insulating layer.

According to another embodiment of the first aspect, the protrusions of the frame are anchor-shaped, preferably wherein each of the pair of suture holes is located between the frame and flukes of the anchor-shaped protrusion.

According to a further embodiment of the first aspect, the frame comprises polymer and/or metal. According to another embodiment of the first aspect, the frame comprises polyetheretherketon (PEEK) or a nickel-titanium alloy.

According to yet another embodiment of the first aspect, the frame has a thickness of 0.15 to 1 mm, preferably of 0.3 to 0.75 mm, more preferably of 0.45 to 0.55 mm.

According to another embodiment of the first aspect, the flat electrode comprises at least 2 pairs of suture holes, at least 3 pairs, at least 4 pairs, at least 5 pairs, at least 6 pairs, at least 7 pairs, or at least 8 pairs, more preferably 6 pairs of suture holes; and at most 16 pairs suture holes, at most 14 pairs, at most 12 pairs, and at most 10 pairs.

In an embodiment of the first aspect, the suture holes have a diameter of 0.5 to 3 mm, preferably of 1 mm to 2 mm, more preferably of 1.3 to 1.7 mm.

According to a further embodiment of the first aspect, the first and second insulating layers comprise silicone, preferably a reinforced silicone comprising an integrated polymer mesh, preferably a PET-mesh.

According to a further embodiment of the first aspect, the first insulating layer comprises perforations passing through the area on which the conductive material is located, preferably the number of perforations being between 10 and 500, more preferably between 30 and 400, even more preferably between 50 and 300, most preferably between 55 and 260.

According to an embodiment of the first aspect, each of the perforations has a diameter of 0.5 to 5 mm, preferably of 1 mm to 4.5 mm, more preferably of 1.5 to 4 mm, even more preferably of 2 to 3.5 mm, most preferably of 2.5 to 3.2 mm.

According to yet another embodiment of the first aspect, the electrical connector is in the form of a button. The button is preferably arranged in one of the distal surface portions of the conductive material. Preferably, the button further comprises an inner button portion on the conductive material side, an outer button portion on the first insulating layer side, and a conductor fixedly sandwiched between the inner and outer button portions.

In a second aspect, the invention further provides a process of manufacturing a flat electrode, comprising the steps of a) preparing a flexible conductive material, a frame, a first insulating layer comprising curable silicone rubber, at least a second insulating layer comprising curable silicone rubber and at least one opening, and suture holes penetrating the first and second insulating layer, b) arranging the conductive material on the first insulating layer and embedding an edge portion thereof into the first insulating layer, c) laminating at least the second insulating layer to the assembly prepared in step b) such that the edge portion of the conductive material is covered by the second insulating layer and a center portion of the conductive material is exposed through the at least one opening of the second insulating layer, and d) subjecting the assembly resulting from step c) to a post-curing treatment.

According to an embodiment of the second aspect, the process further comprises a step of arranging a frame between the first insulating layer and the second insulating layer before laminating in step b). Preferably, the conductive material and the frame are separated from each other by at least one additional insulating layer before laminating in step b).

In a third aspect, the invention further provides an implantable lead assembly comprising an implantable coil electrode, an implantable flat electrode according to the first aspect of the invention, and a control unit, to which the coil electrode and the flat electrode are electrically connected through conductors, wherein the control unit is configured to establish a potential difference between the coil electrode and flat electrode, so that an electric current can flow between the two electrodes, the coil electrode is configured to be positioned in the right ventricle of a heart, and the flat electrode is configured to be positioned on the pericardium of the left ventricle outside of a heart.

### BRIEF DESCRIPTION OF THE DRAWINGS

The present disclosure will be more readily appreciated by reference to the following detailed description when being considered in connection with the accompanying drawings in which:

FIG. 1 is an illustration of a flat electrode according to the invention schematically showing (A) the flat electrode from the conductive surface side, and (B) from the electrically insulated surface side opposite to the conductive surface side.

FIG. 2 is an illustration of a flat electrode according to a preferred embodiment of the invention, schematically showing (A) the flat electrode from the conductive surface side comprising a frame with protrusions, wherein the second insulating layer is removed, and (B) from the electrically insulated surface side on the opposite side of the conductive surface side comprising perforations.

FIG. 3 is a partial illustration of a flat electrode according to a preferred embodiment of the invention comprising a frame schematically showing (A) an i-shaped protrusion between a pair of suture holes, (B) an anchor-shaped protrusion between a pair of suture holes, and (C) a pair of suture holes fixed by a thread passing over the protrusion.

FIG. 4 is a sectional view of a flat electrode according to the invention schematically showing the structure of an electrical connector in the form of a button.

FIG. 5 is an illustration schematically showing an exemplary disposition of an internal coil electrode and an external flat electrode according to a preferred embodiment of the invention in and outside of a heart as internal organ.

### DETAILED DESCRIPTION OF PREFERRED EMBODIMENTS

In the following, the invention will be explained in more detail with reference to the accompanying figures. In the Figures, like elements are denoted by identical reference numerals.

In order that the present description can be more readily understood, certain terms are first defined. Additional definitions are set forth throughout the detailed description.

It is to be noted that the term “a” or “an” entity refers to one or more of that entity; for example, “an opening,” is understood to represent one or more openings. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, Korpas, David.Implantable cardiac devices technology. Berlin: Springer, 2013; Troutman, Leslie. “Dictionary of Medical Technology.” RQ 32.3(1993 ): 421-423, provide one of skill with a general dictionary of many of the terms used in this disclosure.

Units, prefixes, and symbols are denoted in their Système International d'Unités (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or Lower).

The terms “flat electrode”, “flat lead”, “patch electrode”, “patch lead”, or “patch” are used interchangeably herein and refer to a flat implantable medical device. It is to be understood that the design of the flat electrode must ensure the main function thereof, i.e., the distribution of an electrical current through the organic tissue. Accordingly, in general, an implantable flat electrode has at least a conductive surface, an electrically insulated surface on the opposite side, and an electrical connector for coupling to a control/power unit. Such a flat electrode usually comprises flexible biocompatible and biostable materials to fit to the geometry of the target tissue surface in a way so that a contact between the tissue surface and the majority of the flat electrode conductive surface is possible and fastening means to be fixedly attached to the target tissue.

The terms “mounted”, “attached”, “fixed”, “fastened”, or “secured” are used to describe positioning or attaching of the electrode to the organic tissue. In the context of the disclosure, the electrode is attached by being sutured, clamped, stapled or fixed by any other method, preferably attached by being sutured to an organic tissue, such as, for example, to the outside of the left ventricle and/or the outside of the right ventricle of a heart or under and/or onto encapsulating tissue of an organ, e.g. to the pericardium. It may also be possible to position the flat electrode subcutaneously or on the outside of the skin of a subject.

In the context of the invention, the term “implantable flat electrodes” refers to flat electrodes that can be mounted intracorporeally or extracorporeally. The term “organic tissue” refers to external or internal organs. In the context of the disclosure, the organic tissue is preferably an internal organ preferably selected from a group comprising brain, nervous tissue, heart, kidney, liver, stomach, intestine, gallbladder, and pancreas, more preferably selected form a group comprising heart, kidney, and liver, and most preferably is a heart.

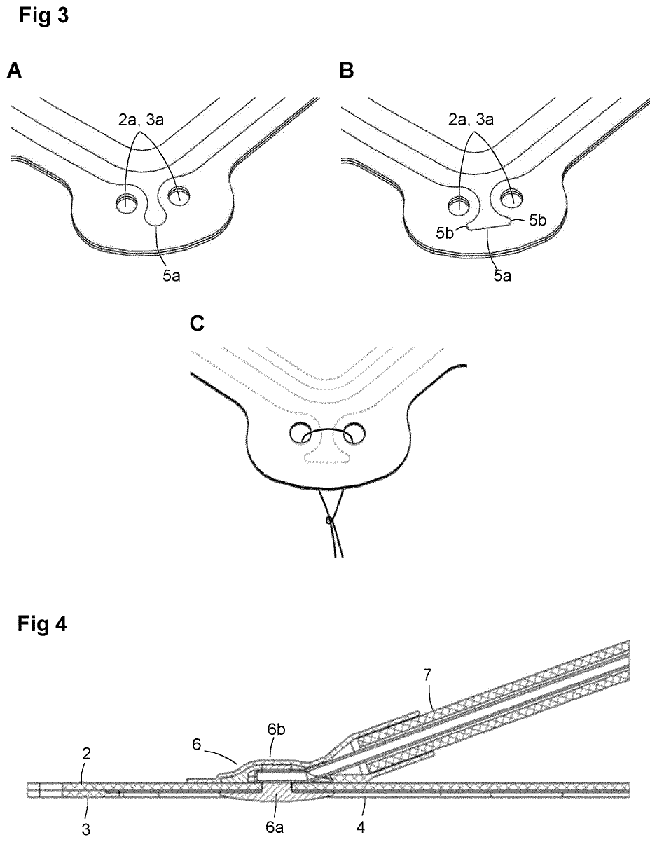

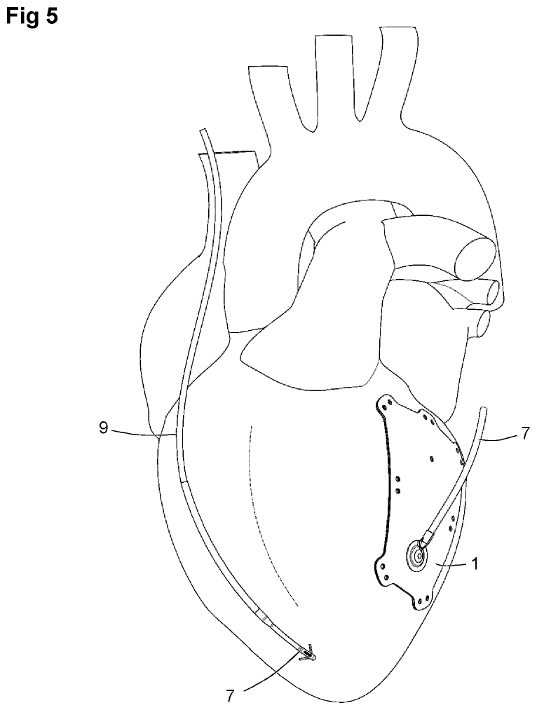

The term “suturing” refers to fixing of the electrode to the target tissue by surgical suture, which, as illustrated in FIG. 3C, substantially comprises a surgical thread passed through the first of the pair of adjacent suture holes, a portion of the tissue surface, and the second of the pair of adjacent suture holes, wherein both ends of the thread are knotted together so that the thread forms a closed loop, thereby attaching the portion of the electrode comprising the pair of adjacent suture holes to the tissue.

The term “biocompatibility” or “biocompatible” describes appropriate biological requirements of a biomaterial or biomaterials used in a medical device as well as the ability of a material to perform with an appropriate host response in a specific application. In the context of the disclosure, the term “biocompatibility” specifically means the ability of the material of the flat electrode 1 to function in vivo without eliciting detrimental local or systemic responses in the body. The term “biostability” or “biostable” refers to the ability of a material to maintain its physical and chemical integrity after implantation into a living tissue.

The term “current” refers to electrical current and may be direct current or alternating current. During the intended medical treatment, the current density at the flat electrode will be adjusted to preferable 0.1 to 100 μA/cm², more preferable to 0.5 to 10 μ/cm².

Accordingly, the present invention provides flat electrodes for treatment of organic tissue with a current, wherein the flat electrode 1 comprises laminated first and second insulating layers 2, 3 comprising an elastic, biocompatible and biostable material, the second insulating layer 3 having at least one opening 3c, a flexible conductive material 4 sandwiched between the first and second insulating layer 2, 3, wherein the conductive material 4 comprises an edge portion 4a embedded into the first insulating layer 2 and covered by the second insulating layer 3, and a center portion 4b of the conductive material 4 exposed through the at least one opening 3c of the second insulating layer 3 and not fixed to the first insulating layer 2, two or more pairs of suture holes 2a, 3a provided in circumferential edge portions 2b, 3b of the first and second insulating layers 2, 3, wherein the edge portions 2b, 3b portions are laminated onto each other without the conductive material 4 sandwiched in between, and an electrical connector 6 electrically coupled to the conductive material 4.

The invention provides following design related benefits: The first insulating layer 2 and the exposed portion 4b of the conductive material 4 provide, due to the two-layered structure, a high flexibility of the electrode 1 in the area of the exposed portion 4b of the conductive material 4 in order to properly adapt to the surface of the organ to be treaded. The circumferential edge portion 2b, 3b comprising at least two layers enhances the rigidity by dimensionally stabilizing the shape of the implanted electrode 1 as compared to a common one-layer structure. Additionally, the at least two-layer structure around the suture holes 2a, 3a in the circumferential edge portion 2b, 3b reinforces the suture holes 2a, 3a against tear-out of suture strings. The sandwiching of an edge portion 4a of the conductive material 4 between the first and second insulating layers 2, 3 all the way around further reinforces the fixation of the conductive material 4 and the edge portion 4a thereof in the flat electrode 1 without the need for an additional element, which reduces a risk of the conductive material 4 detaching from the flat electrode 1 after a certain period following implantation. For this effect, the size of the conductive material 4 is smaller in plan view than size of the first insulating layer 2 and the second insulating layer 3. The embedding of the edge portion 4a of the conductive material 4 into the first insulating layer 2 provides even more flexibility of the electrode 1 in area of the conductive material 4 since a center portion of the conductive material 4 is not being fixed and is thus able to move relative to the first insulating layer 2. The flexibility facilitates implantation of the electrode and allows it to adapt better to the external geometry of the target organ, e.g. a heart.

It is to be understood that further preferred embodiments and particular designs of the flat electrode 1 of the invention can be easily determined by one skilled in the art in accordance with the application purpose and application site thereof within the context of the present invention and based on the common general knowledge and the information given herein.

In an exemplary embodiment of the flat electrode 1, the at least one opening 3c of the second insulating layer 3 is a central opening.

In a preferred embodiment of the flat electrode 1 according to the present invention, the conductive material 4 is embedded in or bonded to the first insulating layer 2 and/or second insulating layer 3, preferably wherein an edge portion 4a of the conductive material 4 is embedded into the first insulating layer 2 and is covered by the second insulating layer 3.

In one preferred embodiment of the flat electrode 1 according to the present invention, the conductive material 4 comprises a metallic mesh. Preferably, the metallic mesh comprises platinum, and more preferably a platinum-iridium alloy. The platinum-iridium alloy preferably has an alloy proportion of platinum-iridium of 70:30 to 99:1, more preferably of 80:20 to 95:15, most preferably of 88:12 to 92:8. Platinum and alloys thereof have inherent corrosive resistance, high biocompatibility, and radiopaque properties, making it a perfect candidate for a range of medical applications. In an exemplary embodiment, the conductive material 4 comprises woven platinum Iridium 90:10 wires, wherein the single wire outer diameter is 43 μm, the mesh pattern is 1 over 1, and the mesh wide is 150 wires/inch.

In a further preferred embodiment of the flat electrode 1 according to the present invention, the conductive material 4 has a waist shape in plan view comprising in the longitudinal direction thereof two distal portions connected by a central waist portion, wherein the ratio of the width w_{d1}, w_{d2} of the distal portions 4d to the width wₘ Of the central waist portion 4c is from 2:1 to 6:1, preferably from 3:1 to 5:1. As indicated in FIG. 1A by arrows, the width w_{d1}, w_{d2} of the distal portions 4d and the width Wm of the waist portion 4c are measured perpendicular to a longitudinal axis of the flat electrode 1. In a preferred embodiment, the specific measurement points shown in FIG. 1A should be used. However, this should not be understood as limiting, and such measurements may be taken at any suitable point of the respective portion. Thus, any waist shape is feasible in which all the ratios between each of the widths of the waist portion 4c and each of the widths of the distal portion 4d are within the ratio of 1.5:1 to 7:1, preferably from 2:1 to 6:1, more preferably from 3:1 to 5:1. Furthermore, the width w_{d1} may be equal to, greater than, or less than the width w_{d2}.

The waist shape according to the embodiment of the present invention allows bending of the flat electrode 1 around two axes perpendicular to each other in the bending surface while preventing interference of individual parts thereof with each other and keeping the mechanical stress on the individual parts of the electrode low. This shape also allows the electrode to adapt to the uneven surface of organic tissue.

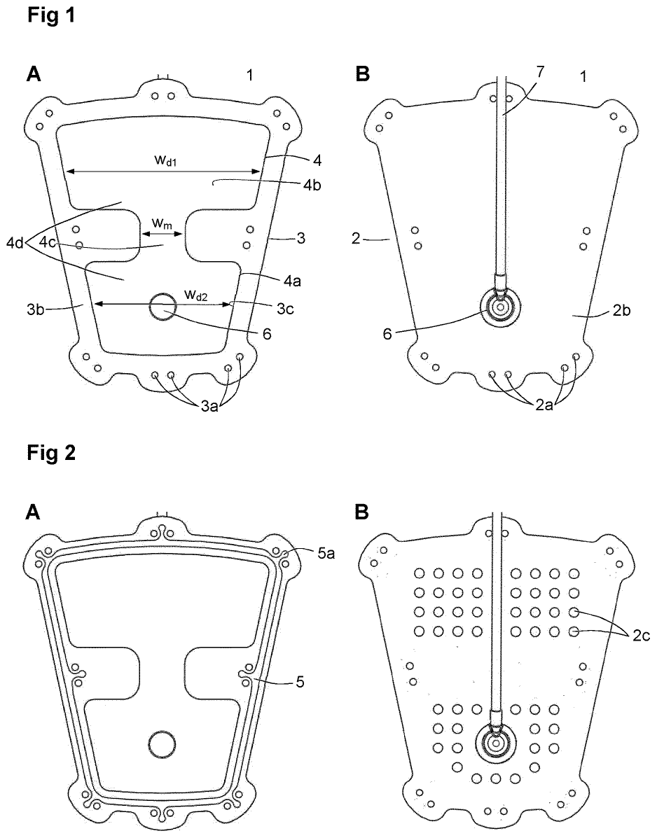

As shown in FIGS. 1 and 2, in a particular embodiment, the central waist portion 4c merges into the distal portions 4d via rounded corners. On both sides between the rounded corners of the opposite distal portions 4d, the central waist portion 4c comprises a linear outer edge. This particular design of the center waist portion 4c prevents a notch effect in the area thereof that can occur due to stress concentration when the flat electrode is deformed, which reduces wear in the area of the center waist portion 4c and thus increases the durability of the conductive material 4. For this effect, any shape is possible in which all ratios between the length of the linear edge and each of the radii of the rounded corner are within the ratio of 1:3 to 6:1, preferably from 1:1 to 4:1, more preferably from 4:3 to 2:1. The ratio between the length of the linear edge and each of the radii of the rounded corner may be 3:2.

In a one preferred embodiment of the flat electrode 1 according to the present invention, the flat electrodes further comprise a frame 5 arranged between the first and second insulating layer 2, 3, wherein the frame 5 comprises a plurality of protrusions 5a each protruding from the frame 5 and extending in parallel to the surface of the insulating layers between a pair of adjacent suture holes 2a, 3a. In a further preferred embodiment, the conductive material 4 and the frame 5 are separated from each other by at least one additional insulating layer.

The inventors surprisingly discovered that, by providing the frame 5 with protrusions 5a each protruding from the frame 5 and extending in parallel to the surface of the insulating layers between a pair of adjacent suture holes 2a, 3a, flat electrode 1 is reinforced by the frame 5 while still allowing sufficient bending so that the electrode 1 is able to lie flush against a typically uneven surface of organic tissue to which it is sutured, and a tensile load applied by the thread to a pair of adjacent suture holes 2a, 3a is largely distributed to and absorbed by the sturdy protrusion 5a, thus significantly improving the durability of the pair of adjacent suture holes 2a, 3a against delamination, deformation, and tear-out while maintaining an appropriate degree of flexibility of the portion of the electrode 1 comprising the pair of adjacent suture holes 2a, 3a. The at least one additional insulating layer provides additional insulation between the frame 5 and the conductive material 4, which is especially beneficial if the frame 5 comprises a conductive material like metal.

In another preferred embodiment of the flat electrode 1 according to the present invention, the protrusions 5a are anchor-shaped, preferably wherein each of the pair of suture holes 2a, 3a is located between the frame 5 and flukes 5b of the anchor-shaped protrusion.

The flukes 5b of the anchor-shaped protrusion 5a additionally reinforce the suture holes 2a, 3a in the direction away from the frame 5. Optionally, different shapes for the protrusions 5a are possible, such as, for example, the protrusions 5a may be i-shaped, hook-shaped, barb-shaped, club-shaped, or cone-shaped, and the shapes of the protrusions 5a of a flat electrode 1 may also differ from one another.

In a further preferred embodiment of the flat electrode 1 according to the present invention, the insulating layers, i.e. the first insulating layers 2, the second insulating layer 3, and/or any additional insulation layer, comprise silicone, preferably a reinforced silicone comprising an integrated polymer mesh as mono-or multifilament, wherein the polymer mesh preferably comprises polyester, PET and/or PETG.

The integrated polymer mesh further enhances the stability of the silicone material, in particular against tear-out. Furthermore, by specifically selecting a particular integrated polymer mesh, the stiffness and strength of the silicone material can be adjusted without having to change the thickness of the flat electrode 1. This allows the mechanical properties of the materials of the flat electrode 1 to be adapted to specific requirements.

In a further preferred embodiment of the flat electrode 1 according to the present invention, the flat electrode 1 comprises at least 2 pairs of suture holes, at least 3 pairs, at least 4 pairs, at least 5 pairs, at least 6 pairs, at least 7 pairs, or at least 8 pairs, more preferably 6 pairs of suture holes; and at most 16 pairs suture holes, at most 14 pairs, at most 12 pairs, and at most 10 pairs.

In a further preferred embodiment of the flat electrode 1 according to the present invention, the suture holes have a diameter of 0.5 to 3 mm, preferably of 1 mm to 2 mm, more preferably of 1.3 to 1.7 mm. In a further preferred embodiment of the flat electrode 1 according to the present invention, the first insulating layer 2 comprises perforations 2c passing through the area on which the conductive material 4 is located, preferably the number of perforations 2c being between 10 and 500, more preferably between 30 and 400, even more preferably between 50 and 300, most preferably between 55 and 260. Each of the perforations 2c preferably has a diameter of 0.5 to 5 mm, more preferably of 1 mm to 4.5 mm, more preferably of 1.5 to 4 mm, even more preferably of 2 to 3.5 mm, most preferably of 2.5 to 3.2 mm.

In rare cases of flat electrodes implanted, for example, on the outside of the heart and having an first insulating layer 2 without perforations, the inventors observed after some time of implantation that fibrotic tissue had grown through the conductive material 4 and an excessive voluminous formation of fibrotic tissue (fibrosis) occurred between the conductive material 4 and the first insulating layer 2 which led to unfavorable deformations and in the worst case to detachment of the flat electrode from the target tissue. When perforations 2c are provided in the first insulating layer 2, fibrotic tissue growing through the conductive material 4 can continue to grow out of the flat electrode through the perforation 2c in the first insulating layer 2, thus preventing the accumulation of voluminous fibrosis between the conductive material 4 and the first insulating layer 2. In this way, the perforations 2c reduce the risk of deformation and detachment of the flat electrode 1. For use in long-term implantable devices, the frame material is required to have a mechanical strength, an appropriate stiffness, biocompatibility, and chemical resistance, formability and stability at high temperature for manufacturing of the electrode 1. By conducting experiments, the inventors have found that metal, specifically metallic alloys such as nickel-titanium alloy, and polymers such as polyimide, high-density polyethylene (HDPE), polytetrafluoroethylene (PTFE), and specifically polyether ether keton (PEEK) exhibit these properties and are thus suited as frame material.

Accordingly, in a preferred embodiment of the flat electrode 1 according to the present invention, the frame 5 comprises polymer such polyimide, HDPE, PTFE, and PEEK, preferably PEEK.

Accordingly, in a preferred embodiment of the flat electrode 1 according to the present invention, the frame 5 comprises metal, preferably a platinum alloy and/or a titanium alloy, most preferably nickel-titanium alloy.

In a further preferred embodiment of the flat electrode 1 according to the present invention, the frame 5 has a thickness of 0.15 to 1 mm, preferably of 0.3 to 0.75 mm, more preferably of 0.45 to 0.55 mm. In a further preferred embodiment of the flat electrode 1 according to the present invention, the electrical connector 6 is in the form of a button. The button preferably is arranged in one of the distal surface portions 4d of the conductive material 4. Preferably the button 6 further comprises an inner button portion 6a on the conductive material side, an outer button portion 6b on the first insulating layer side, and a conductor 7 configured to be connected to a control unit and which is fixedly sandwiched between the inner and outer button portions 6a, 6b. The fixing of conductor 7 between the inner and outer button portions 6a, 6b is preferably carried out by welding, more preferably by laser welding. The outer button portion 6b may further be insulated by a cover, preferably a silicone cover.

The invention further provides a process of manufacturing a flat electrode, comprising a) preparing a flexible conductive material 4, a frame 5, a first insulating layer 2 comprising curable silicone rubber, at least a second insulating layer 3 comprising curable silicone rubber and at least one opening 3c, and suture holes penetrating the first and second insulating layer 2, 3, b) arranging the conductive material 4 on the first insulating layer 2 and embedding an edge portion 4a thereof into the first insulating layer 2, c) laminating at least the second insulating layer 3 to the assembly prepared in step b) such that the edge portion 4a of the conductive material 4 is covered by the second insulating layer 3 and a center portion 4b of the conductive material 4 is exposed through the at least one opening 3c of the second insulating layer 3, and d) subjecting the assembly resulting from step c) to a post-curing treatment.

The use of curable silicone layers that are not fully cross-linked enables adhesive-free bonding in the sandwich structure, including polymer reinforcement of the silicone.

In a preferred embodiment of process of manufacturing a flat electrode, the process further comprises a step of arranging a frame 5 between the first insulating layer 2 and the second insulating layer 3 before laminating in step b). The conductive material 4 and the frame 5 are further preferably separated from each other by at least one additional insulating layer before laminating in step b). This at least one additional insulating layer provides additional insulation between the frame 5 and the conductive material 4, which is especially beneficial when the frame 5 comprises conductive metal.

In an exemplary embodiment of the process of manufacturing, first and second insulating layers 2, 3 both comprising a curable silicone rubber were cut using a 2D cutting table and/or punches. The contours of first and second insulating layers 2, 3, the suture holes 2a, 3a, the central opening 3c and the perforations 2c are cut for each layer by control of a cut programmable file on the 2d cutting table and/or are cut using a specific punching tool. As the conductive material 4, a platinum-iridium alloy (Pt—Ir) mesh which is woven 1 over 1, has an alloy proportion of platinum-iridium of 90:10, a 40 μm wire thickness, and a 150 mesh is contour cut by laser cutting. A frame 5 is cut from PEEK by waterjet cutting having a thickness of 0.5 mm.

The Pt—Ir mesh is circumferentially welded with a contour sealing plate by pressing the cut wire ends thereof into the uncured silicone material of the first insulating layer 2 at around 5 bar and around 120° C. thereby fixing the wire ends to the first insulation layer 2.

In the following, the PEEK frame 5 is placed around the circumferential weld of the fixed wire ends, and the second insulating layer 3 is placed on the first insulating layer 2, the PEEK frame 5, and the circumferentially weld of the Pt—Ir-Mesh and sealed to the circumferentially weld with a pressure plate at around 5 bar and around 120° C. such that a center portion 4b of the Pt—Ir-Mesh is exposed through the central opening 3c of the second insulating layer 3 and the second insulating layer 3 covers the circumferentially weld, resulting in a layered electrode assembly.

In a last step, the assembly is positioned in a curing oven and heated, wherein the electrode assembly is post-cured such that the silicone material is vulcanized throughout.

It would also be conceivable that, instead of welding or vulcanization, a bonding process or molding by silicone injection molding may be performed.

The invention further provides an implantable lead assembly comprising an implantable coil electrode 8, an implantable flat electrode 1 according to the embodiments of the invention, and a control unit, to which the coil electrode 8 and the flat electrode 1 are electrically connected through connector lines 7, 9, wherein the control unit is configured to establish a potential difference between the coil electrode 8 and the flat electrode 1, so that an electric current can flow between the two electrodes 1, 8, the coil electrode 8 is configured to be positioned inside the right ventricle of a heart, and the flat electrode 1 is configured to be positioned on the pericardium of the left ventricle outside of the heart.

According to a preferred embodiment of the invention, the implantable electrode assembly is configured for applying a microcurrent between the flat electrode 1 and coil electrode 8 to the heart, preferably for treating heart failure. To improve cardiac function in patients with heart failure by applying microcurrent together with an electric field directly to the heart, the preferred current density is between 1.5 and 10 μA/cm². All embodiments of the present invention as described herein are deemed to be combinable in any combination unless the skilled person considers such a combination to not make any technical sense.

### LIST OF REFERENCE NUMERALS

Flat electrode 1
First insulating layer 2
Second insulating layer 3
Suture hole 2a, 3a
Circumferential edge portion 2b, 3b
Perforation 2c
Central opening 3c
Conductive material 4
Edge portion 4a
Center portion 4b
Waist portion 4c
Distal portion 4d
Frame 5
Protrusion 5a
Fluke 5b
Electrical connector 6
Inner button portion 6a
Outer button portion 6b
Conductor 7, 9
Coil electrode 8

1. A flat electrode for treatment of organic tissue with an electrical current, wherein the flat electrode comprises:
laminated first and second insulating layers (2, 3) comprising an elastic, biocompatible and biostable material, the second insulating (3) layer having at least one opening (3c);
a flexible conductive material (4) sandwiched between the first and second insulating layers (2, 3), wherein the flexible conductive material (4) comprises:
an edge portion (4a) embedded into the first insulating layer (2) and covered by the second insulating layer (3), and
a center portion (4b) exposed through the at least one opening (3c) of the second insulating layer (3) and not fixed to the first insulating layer (2);
two or more pairs of suture holes (2a, 3a) provided in circumferential edge portions (2b, 3b) of the first and second insulating layers (2, 3), wherein the edge portions (2b, 3b) are laminated onto each other without the conductive material (4) sandwiched in between; and
an electrical connector (6) electrically coupled to the conductive material (4).

2. The flat electrode according to claim 1, wherein the conductive material (4) comprises a metallic mesh, preferably wherein the metallic mesh comprises platinum, more preferably a platinum-iridium alloy, the platinum-iridium alloy preferably having an alloy proportion of platinum-iridium of 70:30 to 99:1, more preferably of 80:20 to 95:15, most preferably of 88:12 to 92:8.

3. The flat electrode according to any of claims 1 or 2, wherein the conductive material (4) has a waist shape in plan view comprising in the longitudinal direction thereof two distal portions (4d) connected by a waist portion (4c) therebetween, the ratio of the width perpendicular to the longitudinal direction of the distal portion (4d) to the waist portion (4c) is from 1.5:1 to 7:1, preferably from 2:1 to 6:1, more preferably from 3:1 to 5:1.

4. The flat electrode according to any of claims 1 to 3, further comprising a frame (5) arranged between the first and second insulating layer (2, 3), wherein the frame (5) comprises a plurality of protrusions (5a) each protruding from the frame (5) and extending in parallel to the surface of the insulating layers between a pair of adjacent suture holes (2a, 3a), optionally wherein the frame (5) is separated from the conductive material (4) by at least one additional insulating layer.

5. The flat electrode according to claim 4, wherein the protrusions (5a) are anchor-shaped, preferably wherein each of the pair of suture holes (2a, 3a) is located between the frame (5) and flukes (5b) of the anchor-shaped protrusion.

6. The flat electrode according to claim 4 or 5, wherein the frame (5) comprises polymer and/or metal.

7. The flat electrode according to claim 6, wherein the frame (5) comprises polyether ether keton (PEEK) or a nickel-titanium alloy.

8. The flat electrode according to any of claims 4 to 7, wherein the frame (5) has a thickness of 0.15 to 1 mm, preferably of 0.3 to 0.75 mm, more preferably of 0.45 to 0.55 mm.

9. The flat electrode according to any of claims 1 to 8, further comprising at least 2 pairs of suture holes (2a, 3a), at least 3 pairs, at least 4 pairs, at least 5 pairs, at least 6 pairs, at least 7 pairs, or at least 8 pairs, more preferably 6 pairs of suture holes (2a, 3a); and at most 16 pairs suture holes (2a, 3a), at most 14 pairs, at most 12 pairs, and at most 10 pairs.

10. The flat electrode according to any of claims 1 to 9, wherein the suture holes (2a, 3a) have a diameter of 0.5 to 3 mm, preferably of 1 mm to 2 mm, more preferably of 1.3 to 1.7 mm.

11. The flat electrode according to any of claims 1 to 10, wherein the insulating layers comprise silicone, preferably a reinforced silicone comprising an integrated polymer mesh, preferably a PET-mesh.

12. The flat electrode according to any of claims 1 to 11, wherein the first insulating layer (2) comprises perforations (2c) passing through the area on which the conductive material (4) is located, preferably the number of perforations (2) being between 10 and 500, more preferably between 30 and 400, even more preferably between 50 and 300, most preferably between 55 and 260.

13. The flat electrode according to claim 12, wherein each of the perforations (2) has a diameter of 0.5 to 5 mm, preferably of 1 mm to 4.5 mm, more preferably of 1.5 to 4 mm, even more preferably of 2 to 3.5 mm, most preferably of 2.5 to 3.2 mm.

14. The flat electrode according to any of claims 1 to 13, wherein the electrical connector (6) is in the form of a button; preferably wherein
the button is arranged in one of the distal portions (4d) of the conductive material (4); and
preferably wherein further the button (6) comprises:
an inner button portion (6a) on the conductive material side, an outer button portion (6b) on the first insulating layer side, and a conductor (7) fixedly sandwiched between the inner and outer button portions (6a, 6b).

15. A process of manufacturing the flat electrode as defined in any of claims 1 to 14, wherein the process comprises the following steps:
a) preparing a flexible conductive material (4), a frame (5), a first insulating layer (2) comprising curable silicone rubber, at least a second insulating layer (3) comprising curable silicone rubber and at least one opening (3c), and suture holes (2a, 3a) penetrating the first and second insulating layer (2, 3);
b) arranging the conductive material (4) on the first insulating layer (2) and embedding an edge portion (4a) thereof into the first insulating layer (2);
c) laminating at least the second insulating layer (3) to the assembly prepared in step b) such that the edge portion (4a) of the conductive material (4) is covered by the second insulating layer (3) and a center portion (4b) of the conductive material is exposed through the at least one opening (3c) of the second insulating layer (3); and
d) subjecting the assembly resulting from step c) to a post-curing treatment.

16. The process of manufacturing the flat electrode according to claim 15, wherein the process further comprises a step of arranging a frame (5) between the first insulating layer (2) and the second insulating layer (3) before laminating in step b), optionally wherein the conductive material (4) and the frame (5) are separated from each other by at least one additional insulating layer before laminating in step b).

17. An implantable lead assembly comprising an implantable coil electrode (8), an implantable flat electrode (1) according to any of claims 1 to 14, and a control unit, to which the coil electrode (8) and the flat electrode (1) are electrically connected through conductors (7, 9), wherein
the control unit is configured to establish a potential difference between the coil electrode (8) and flat electrode (1), so that an electric current can flow between the two electrodes (1, 8),
the coil electrode (8) is configured to be positioned in the right ventricle of a heart, and
the flat electrode (1) is configured to be positioned on the pericardium of the left ventricle outside of a heart.
